(19) United States
(12) Patent Application Publication
(10) Pub. No.: US 2018/0153720 A1
(43) Pub. Date: Jun. 7, 2018

(54) STENT DELIVERY ASSEMBLY

(71) Applicant: Cook Medical Technologies LLC, Bloomington, IN (US)

(72) Inventors: Woong Kim, West Lafayette, IN (US); Manjiri Dhoke, West Lafayette, IN (US)

(73) Assignee: Cook Medical Technologies LLC, Bloomington, IN (US)

(21) Appl. No.: 15/825,558

(22) Filed: Nov. 29, 2017

Related U.S. Application Data
(60) Provisional application No. 62/429,202, filed on Dec. 2, 2016.

(51) Int. Cl. A61F 2/958; A61M 25/10; A61F 2/856; A61F 2/848

(52) U.S. Cl. CPC A61F 2/958; A61M 25/1002; A61B 17/22032; A61F 2/848; A61M 2025/1086; A61F 2/856

(57) ABSTRACT

A stent delivery assembly includes a delivery balloon having a balloon surface with microbristles extending therefrom, wherein at least a portion of the microbristles has a length within a range of 0.8 through 1.2 mm. Another stent delivery assembly includes a delivery balloon having a balloon surface with microbristles extending therefrom and a stent disposed around the delivery balloon, wherein the stent has interconnected struts with a strut thickness and the delivery balloon has a balloon surface with microbristles extending therefrom, wherein at least a portion of the microbristles has a length that is greater than the strut thickness. The balloon surface may form a monolithic structure with the microbristles. The microbristles may be made of a stiffer material than the balloon surface.

### TECHNICAL FIELD

The present application deals with a stent delivery assembly, in particular a stent delivery assembly including a delivery balloon.

### BACKGROUND

A stent is a generally cylindrical prosthesis introduced via a catheter into a lumen of a body vessel in a collapsed configuration having a generally reduced diameter and then expanded to the diameter of the vessel. In the expanded configuration, the stent supports and reinforces the vessel walls while maintaining the vessel in an open, unobstructed condition. Stents may be self-expanding or balloon-expandable. Balloon expandable stents are expanded by placing the stent on a deflated balloon catheter and by inflating the balloon at the location where the stent is to be placed.

It has been observed that, during the inflation of the balloon, the stent may move from its initial crimped location with respect to the balloon. Segmented stents have segments configured to detach from one another to allow for flexible positioning of each segment, especially in vessels having tortuous anatomy in the implant location. During the delivery and inflation process, these segments might additionally shift relative to one another longitudinal to the balloon.

### SUMMARY

According to a first aspect of the present application, a stent delivery assembly includes a delivery balloon having a balloon surface with microbristles extending therefrom, wherein at least a portion of the microbristles has a length within a range of 0.8 through 1.2 mm. This length provides for a secure placement on the balloon without adding excessive bulk to the balloon in a collapsed state.

According to another aspect, the microbristles may have a thickness within a range of 0.08 mm through 0.12 mm for optimum stiffness and flexibility.

According to a further aspect, by arranging individual microbristles in locations that are axially spaced apart by 0.5 mm through 0.7 mm, at least a subset of the microbristles will extend through gaps between stent struts for engaging the stent. Under a similar rationale, the microbristles may be individually arranged in locations that are circumferentially spaced apart by 0.4 mm through 0.5 mm.

According to one aspect of the disclosure, the balloon surface may form a monolithic structure with the microbristles.

According to another aspect of the disclosure, the microbristles may be made of a stiffer material than the balloon surface.

According to a further aspect, the balloon has a collapsed state, in which 280 through 500 microbristles per cm2 may extend from the balloon surface, in particular 340 to 400 microbristles per cm2.

According to yet another aspect, for promoting a penetration of the stent by the microbristles, the balloon surface may include molded base nipples in locations from which the microbristles extend.

According to one aspect, for making the balloon suitable for a variety of different stents, the microbristles may have irregular distances from one another.

According to another aspect, a stent delivery assembly includes a delivery balloon and a stent disposed around the delivery balloon, wherein the stent has interconnected struts with a strut thickness and the delivery balloon has a balloon surface with microbristles extending therefrom. At least a portion of the microbristles has a length that is greater than the strut thickness.

According to a further aspect, the microbristles have a greater flexibility than the struts so that the microbristles bend around the struts.

According to yet another aspect, the microbristles have a length of 0.8 through 1.2 mm and the strut thickness is within a range of 0.2 through 0.3 mm.

Further details and benefits of the present disclosure become apparent from the following detailed description by way of the accompanying drawings.

The drawings are provided herewith for purely illustrative purposes and are not intended to limit the scope of the present invention.

### BRIEF DESCRIPTION OF THE DRAWINGS

In the drawings,

FIG. 1a shows a stent delivery assembly including a delivery balloon and a stent in a collapsed configuration prior to delivery;

FIG. 1b shows a close-up detail of FIG. 1a;

FIG. 1c shows the close-up detail of FIG. 1b after crimping;

FIG. 2 shows the stent delivery assembly of FIG. 1a in a first partially expanded configuration;

FIG. 3a shows the stent delivery assembly of FIG. 1a in a second partially expanded configuration;

FIG. 3b shows a close-up detail of FIG. 1a;

FIG. 4 shows the stent delivery assembly of FIG. 1a in a fully expanded configuration;

FIG. 5a shows a schematic view of relative positions of microbristles relative to stent struts in a stent delivery assembly of FIG. 4;

FIG. 5b shows a close-up detail of FIG. 5a;

FIG. 6 shows the stent delivery assembly of FIG. 1a during balloon deflation;

FIG. 7 shows a close-up detail of a balloon surface according to a first embodiment; and

FIG. 8 shows a close-up detail of a balloon surface according to a second embodiment.

### DETAILED DESCRIPTION OF THE DRAWINGS

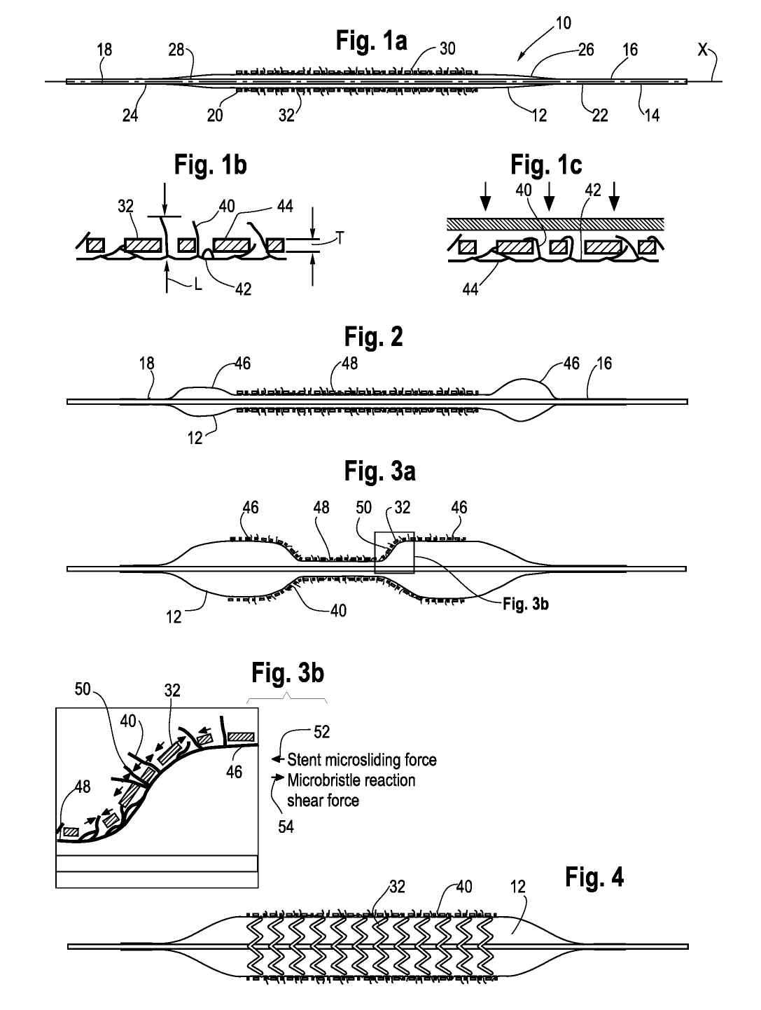

Referring to FIG. 1a, a stent delivery assembly 10 is shown in a collapsed state. A stent delivery balloon 12 surrounds an inner tube 14 that defines a longitudinal axis X and extends beyond a proximal end 16 of the delivery balloon 12 on one side and beyond a distal end 18 of the delivery balloon 12 on the other side. An inflation lumen for delivering saline solution into the annular space 20 surrounding the inner tube 14 in the interior volume of the delivery balloon 12 may be formed as a second lumen inside the inner tube 14 with a radial opening into the annular space 20. Alternatively, the proximal end 16 of the delivery balloon 12 may be affixed to an outer tube (not shown) that terminates in the annular space 20. Generally, any known arrangement to inflate the delivery balloon 12 is suited for obtaining the benefits of the present disclosure.

The delivery balloon 12 is composed of generally five sections 22, 24, 26, 28, and 30. At the proximal end 16, the delivery balloon 12 includes a proximal attachment neck 22 for sealingly affixing the proximal end 16 to the inner tube 14 (or to the outer tube if present). At the distal end 18, the delivery balloon 12 includes a distal attachment neck 24 for sealingly affixing the distal end 18 to the inner tube 14.

Adjacent to the proximal attachment neck 22, the delivery balloon 12 includes a proximal tapered portion 26, and adjacent the distal attachment neck 24, the delivery balloon 12 includes a distal tapered portion 28. Each tapered portion has an increasing circumference with increasing distance from the respective adjacent proximal attachment neck 22 or distal attachment neck 24.

Centrally arranged between the proximal tapered portion 26 and the distal tapered portion 28, the delivery balloon 12 includes a tubular central portion 30 connecting the proximal tapered portion 26 and the distal tapered portion 28.

The central portion 30 carries a tubular, radially expandable stent 32 forming an arrangement of struts 44. Without limitation, the stent 32 may be of a one-piece construction or a segmented stent 32 formed from axially aligned tubular segments 34 that each occupy. The segments 34 may be connected in the collapsed state via connectors 36, 38 between neighboring stent segments 34. The connectors 36, 38 may open during the expansion of the stent 32 to release the neighboring segments 34 from one another.

Along the central portion 30, the surface of the delivery balloon 12 carries a plurality of tiny bristles 40 that will in the following be called microbristles 40. As can be seen from the close-up detail view of FIG. 1b, the microbristles 40 have a length L that exceeds the radial thickness T of the struts 44 of the stent 32. The length of the microbristles 40, however, is limited to a length L that keeps the microbristles 40 from intertwining with the microbristles 40 in surrounding locations. This does not mean that the distance between axially spaced microbristles 40 is greater than the length L, but that the length L makes it unlikely that the microbristles 40 form twists or knots with neighboring microbristles 40. The maximum feasible length L depends, for example, on the stiffness and thickness T of the microbristles 40. The stiffer the microbristles 40 are, the less likely they are to form knots. Also, the thicker the microbristles 40 are, the longer they can be because a longer length L is required to loop around a neighboring microbristle 40. In any event, the microbristles 40 have a greater flexibility, i.e. are softer, than the struts 44 so that the microbristles 40 deform without deforming the struts 44.

In one example, the microbristles 40 may have a length L of 0.8 through 1.2 mm if the thickness T of the stent struts 44 is within a range of 0.2 through 0.3 mm. The microbristles 40 may further have a bristle thickness within the range of 0.08 mm through 0.12 mm. The individual microbristles 40 may be arranged as single filaments in locations that are axially spaced apart by 0.5 mm through 0.7 mm and circumferentially by 0.4 mm through 0.5 mm when the delivery balloon 12 is in the collapsed constellation. Upon expansion of the delivery balloon 12, at least the circumferential spacing will increase proportionally with the balloon circumference.

These measurements may, for example, define a surface density of the microbristles 40 of 280 through 500 microbristles 40 per cm2, preferably 340 to 400 microbristles 40 per cm2.

As further visible in FIG. 1b, the microbristles 40 are in part buried under the stent 32, which presses them against the balloon surface 42, and in part penetrate the stent 32 to extend radially outward through gaps between the stent struts 44. As a result, as shown in FIG. 1c, because the microbristles 40 have a length L that is greater than the thickness T of the stent struts 44, the microbristles 40 that extend radially outward are bent and folded over the stent struts 44 on the outer surface of the stent 32 upon crimping the assembly 10 for packaging. This generates retention forces counteracting any longitudinal shifting of the stent 32 relative to the delivery balloon 12 in the form of shear forces.

The process of moving the stent delivery assembly 10 to an intended implant location is generally known and will not be discussed in further detail. After the collapsed stent delivery assembly 10 is placed in the intended implant location, the delivery balloon 12 is expanded with saline solution introduced into the annular space 20 of the inner balloon volume under pressure sufficient to expand the delivery balloon 12 against resistive forces in the stent 32 and against prevailing surrounding pressure at the implant location.

Because the proximal tapered portion 26 and the distal tapered portion 28 are free from restraint by the stent 32, these two portions 26 and 28 will expand first as shown in FIG. 2. As the volume of the saline solution in the annular space 20 increases, the length of expanded areas 46 of the delivery balloon 12 increases until the expanded areas 46, growing toward each other from both axial sides, reach the central portion 30 bearing the stent 32 and the microbristles 40.

FIG. 3 shows the expansion process progressing into the central portion 30. While, adjacent to the tapered portions, the central portion 30 is already expanded, a collapsed area 48 remains in the center. This causes a large gradient in the balloon circumference within the central portion 30, which results in a steep slope 50. The slope 50 is shown in greater detail in FIG. 3b. Due to the resistance of the stent 32 against the expansion, a longitudinal microsliding force 52 acts on the stent 32 toward the central, collapsed area 48, where the stent 32 can still temporarily maintain its collapsed configuration. The microbristles 40, however, react with a counterforce 54 acting on the stent 32. The counterforce 54 is opposed to the microsliding force 52. The counterforce 54 is a shear force 54 generated by the resistance of the portion of microbristles 40 that extend outward through the gaps between the stent struts 44. Each of the microbristles 40 has an inherent stiffness determined by the microbristle material and by the thickness of the microbristle 40. This stiffness first would have to be collectively overcome by the microsliding force 52 before the stent 32 can slide by more than an initial small fraction of a millimeter that builds up the counterforce 54. By selecting a suitable pattern of microbristle 40 locations, the overall number of penetrating microbristles can be optimized.

The stent delivery assembly 10 expands to an expanded state, in which the stent 32 maintains an axially even spaced structure as schematically shown in FIG. 4. This is of particular benefit for segmented stents 32 with axially aligned ring segments 34 that disconnect from one another during balloon expansion.

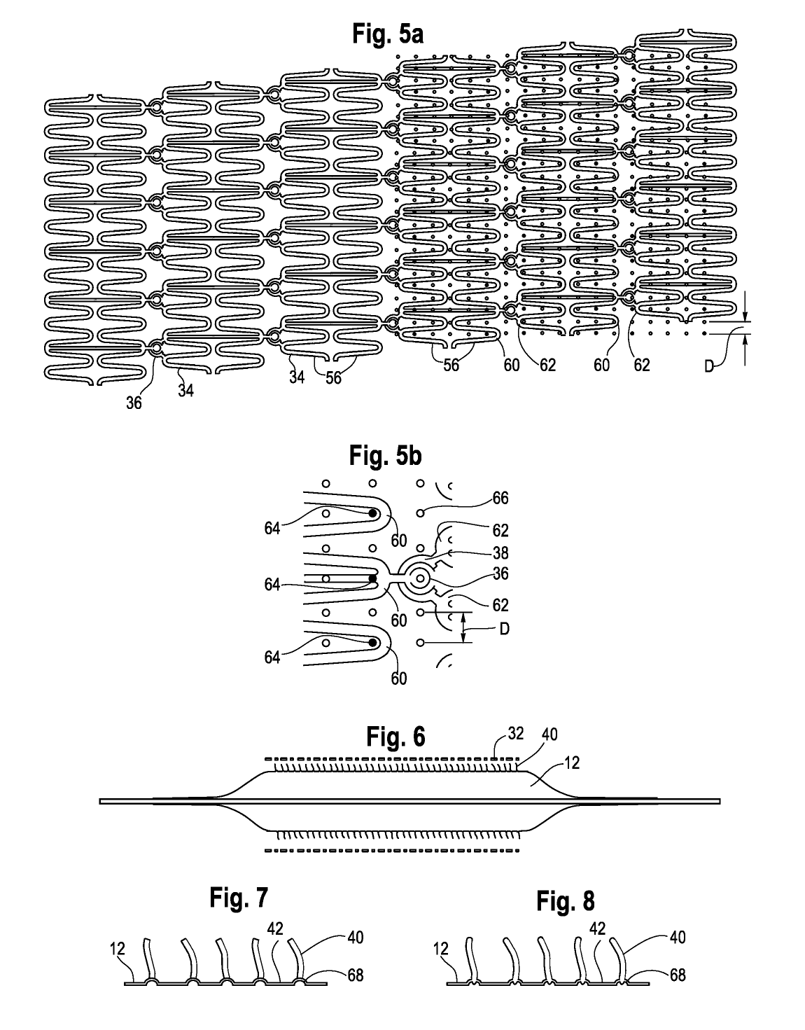

An example of a segmented stent 32 placed on a balloon with microbristles 40 is schematically shown in FIGS. 5a and 5b. FIG. 5b shows a detail of FIG. 5a. The segmented stent 32 is composed of a plurality of stent segments 34, of which six are shown in FIG. 5a. The number of segments 34 may vary between 4 and 30. Each segment 34 includes two serpentined zigzag rings 56 connected via axial struts 58. The axial struts 58 connect the zigzag rings 56 in locations where each of the zigzag rings 56 has a bend 60, 62 remote from the other zigzag ring 56 so that, even when the stent segments 34 undergoes a radial expansion, the axial length of the stent segment 34 remains constant. For example, the number of axial struts 58 may be half the number of remote bends 60, which is a quarter of the total number of bends of an individual zigzag ring 56.

For a more random engagement of the delivery balloon 12 by the microbristles 40, the microbristles 40 may be spaced apart in irregular intervals so that, regardless of the location and structure of the stent 32 disposed on the delivery balloon 12, some of the microbristles 40 will extend through gaps in the stent 32, and a portion of the microbristles 40 will engage the stent 32. Such arrangement of the microbristles 40 provides a more versatile balloon that is suited for a variety of different stents 32.

Adjacent stent segments 34 are connected in a positively locking manner via connector pairs 36, 38 consisting of a male connector 36 and a female connector 38. The male connector 36 extends axially toward the female connector 38 of the adjacent stent segment 34 as an extension of the axial strut 58. The female connector 38 is formed as a pair of clamp arms. Each of the two clamp arms is formed as an extension of one of two adjacent bends 62 of the zigzag ring 56 closest to the male connector 36. In the shown collapsed constellation, the clamp arms of the female connector 38 are spaced circumferentially at such a close distance from one another that they hold the male connector 36 between them as is best evident from FIG. 5b.

Each stent segment 34 has male connectors 36 extending from one axial side and female connectors 38 extending from the opposite axial side so that any number of stent segments 34 can be connected to form a tubular segmented stent 32. The number of male and female connectors 36 and 38 corresponds to the number of axial struts 58, but may be smaller so that not every axial strut 58 extends to a male connector 36. Also, the number of axial struts 58 may be increased or reduced so that every outer bend 60, 62 has an axial strut 58 or only every third or fourth bend 60, 62 has an axial strut 58. These variations depend on the size of the segmented stent 32 and the required rigidity in the collapsed state.

As indicated in the right half of FIG. 5a and in closer detail in FIG. 5b, the locations of the microbristles 40 are spaced apart in a circumferential direction by a smaller distance D than adjacent bends 62 of the stent segments 34. For example, the circumferential distance D of the locations of the microbristles 40 may be chosen to be approximately twice as large as the number of adjacent bends 62. This ensures that at least one location of microbristles 40 is positioned within every bend 60, 62 of the zigzag rings 56, close to the curved portion, both in bends 60 on the side of the male connectors 36 and in opposite bends 62 on the side of the female connectors 38. Axially, the number of locations of the microbristles 40 may lie in the ranges of 4 through 8. In the example of FIG. 5a, approximately 13 locations are distributed over the length L of two stent segments 34. This ensures that a large number of microbristles 40 extends radially through gaps in the stent segments 34.

As shown in FIG. 5b, in the darker locations 64 of the microbristles 40 engage with the stent 32, while in the lighter colored locations 66 the microbristles 40 are not in contact with the stent 32 and thus will not exert shear forces on the stent 32—unless an initial small sliding movement brings further microbristles 40 into engagement with the stent 32. Where the microbristle 40 appears to be buried under an axial strut 58, for example in the center of FIG. 5b, tests have shown that such microbristles 40 will in fact find a path outward past the axial strut 58 on either circumferential side of the axial strut 58.

Now referring to FIG. 6, after the stent 32 has been positioned and expanded to engage the vessel wall, the delivery balloon 12 is deflated so that the microbristles 40 withdraw radially inward from the stent 32.

FIGS. 7 and 8 show options of attaching the microbristles 40 to or of forming the microbristles 40 on the balloon surface 42. In FIG. 7, the microbristles 40 may be made of a different material than the delivery balloon 12 itself. For example, the microbristles 40 may be made of a stiffer material than the balloon surface 42 of the delivery balloon 12 so that the microbristles 40 can have a smaller diameter compared to microbristles 40 that are made of the balloon material. In a non-limiting example, the delivery balloon 12 may be formed of aliphatic or semi-aromatic polyamide (e.g. Nylon), and the microbristles 40 may be made of a stiff polymeric fiber. In FIG. 7, the delivery balloon 12 is formed with base nipples 68 that result from a correspondingly shaped balloon mold. The microbristles 40 are then attached to the outermost portion of each base nipple 68 by an adhesive or heat bonding or by any other suitable method. The base nipples 68 provide flexible adhesion sites when the delivery balloon 12 is not inflated; but once the delivery balloon 12 inflates, the pressure provides additional stiffness to the microbristles 40 so that the base nipples 68 promote erection of the microbristles 40 from the balloon surface 42.

In FIG. 8, the microbristles 40 are molded onto the balloon surface 42 and thus consist of the same material as the balloon surface 42 of the delivery balloon 12. The monolithic structure of FIG. 8 reduces the number of manufacturing steps. In FIG. 8, the base nipples 68 are shown to be much smaller than in FIG. 7.

The base nipples 68 may be omitted entirely where the balloon material and the microbristles 40 have a stiffness that is sufficient to erect the microbristles 40 upon expansion. In addition to the base nipples 68 or as an alternative, for example, the microbristles 40 may be thicker at their base directly adjacent the balloon surface 42 than at their tips for increased stiffness near the balloon surface 42 relative to the tips of the microbristles 40. For example, the microbristles 40 may have a steadily decreasing thickness from the base to the tip or from the base along only a portion of their length L so that the portion closest to the balloon surface 42 is tapered. Alternatively or additionally to the taper or the base nipples 68, the balloon skin may be thickened in localized spots where the microbristles 40 extend from the balloon surface 42.

While the above description constitutes the preferred embodiments of the present invention, it will be appreciated that the invention is susceptible to modification, variation and change without departing from the proper scope and fair meaning of the accompanying claims.

What is claimed is:

1. A stent delivery assembly comprising
a delivery balloon having a balloon surface with microbristles extending therefrom, wherein at least a portion of the microbristles has a length within a range of 0.8 through 1.2 mm.

2. The stent delivery assembly of claim 1, wherein the microbristles have a thickness within a range of 0.08 mm through 0.12 mm.

3. The stent delivery assembly of claim 1, wherein the delivery balloon defines a balloon axis and the microbristles are individually arranged in locations that are axially spaced apart by 0.5 mm through 0.7 mm.

4. The stent delivery assembly of claim 1, wherein the delivery balloon defines a balloon axis and the microbristles are individually arranged in locations that are circumferentially spaced apart by 0.4 mm through 0.5 mm.

5. The stent delivery assembly of claim 1, wherein the balloon surface forms a monolithic structure with the microbristles.

6. The stent delivery assembly of claim 1, wherein the microbristles are made of a stiffer material than the balloon surface.

7. The stent delivery assembly of claim 1, wherein the balloon has a collapsed state, in which 280 through 500 microbristles per cm² extend from the balloon surface.

8. The stent delivery assembly of claim 7, wherein the balloon has a collapsed state, in which 340 to 400 microbristles per cm² extend from the balloon surface.

9. The stent delivery assembly of claim 1, wherein the balloon surface comprises molded base nipples in locations from which the microbristles extend.

10. The stent delivery assembly of claim 1, wherein the microbristles have irregular distances from one another.

11. A stent delivery assembly including
a delivery balloon and
a stent disposed around the delivery balloon,
wherein the stent has interconnected struts with a strut thickness and the delivery balloon has a balloon surface with microbristles extending therefrom, wherein at least a portion of the microbristles has a length that is greater than the strut thickness.

12. The stent delivery assembly of claim 11, wherein the microbristles extend from the balloon surface from locations having irregular distances from one another.

13. The stent delivery assembly of claim 11, wherein the microbristles have a greater flexibility than the struts.

14. The stent delivery assembly of claim 11, wherein the microbristles have a length of 0.8 through 1.2 mm and the strut thickness is within a range of 0.2 through 0.3 mm.

15. The stent delivery assembly of claim 11, wherein the delivery balloon defines a balloon axis and the microbristles are individually arranged in locations that are axially spaced apart by 0.5 mm through 0.7 mm.

16. The stent delivery assembly of claim 11, wherein the delivery balloon defines a balloon axis and the microbristles are individually arranged in locations that are circumferentially spaced apart by 0.4 mm through 0.5 mm.

17. The stent delivery assembly of claim 11, wherein the balloon surface forms a monolithic structure with the microbristles.

18. The stent delivery assembly of claim 1, wherein the microbristles are made of a stiffer material than the balloon surface.

19. The stent delivery assembly of claim 10, wherein the balloon has a collapsed state, in which 280 through 500 microbristles per cm² extend from the balloon surface.

20. The stent delivery assembly of claim 10, wherein the balloon surface comprises molded base nipples in locations from which the microbristles extend.
